(19) United States
(12) Patent Application Publication
(10) Pub. No.: US 2013/0267834 A1
(43) Pub. Date: Oct. 10, 2013

(54) Needle guidance apparatus and method

(71) Applicant: Tracy Scott McGee, Leesburg, GA (US)

(72) Inventor: Tracy Scott McGee, Leesburg, GA (US)

(21) Appl. No.: 13/506,251

(22) Filed: Apr. 6, 2012

(51) Int. Cl. A61B 6/00; A61M 5/00

(52) U.S. Cl. 600/424; 604/272

(57) ABSTRACT

A mechanism for aligning a hypodermic needle or other probe with a predetermined location within a patient has a radio-transparent guide with a lengthwise slot for accommodating the needle. A pair of radiopaque rings spaced at opposite ends of the guide enables the surgeon to align the needle with the predetermined location and advance the needle in a continuous and steady movement toward the predetermined location in spite of the intermittent activation and deactivation of a two-dimensional x-ray apparatus. A slot in each of the rings, moreover, forming gaps in alignment with the lengthwise guide slot permit the guide to be removed as the needle approaches the predetermined location.

### CROSS-REFERENCES TO RELATED APPLICATIONS

None

### FEDERALLY SPONSORED RESEARCH OR DEVELOPMENT

None

### NAMES OF PARTNERS TO A JOINT RESEARCH AGREEMENT

None

### REFERENCE TO “SEQUENCE LISTING”

None

### BACKGROUND OF THE INVENTION

1. Field of the Invention

This invention relates to apparatus and methods for guiding a needle to a predetermined location while at intervals monitoring the progress of that needle toward the location through x-rays and more particularly, to a radio-transparent guide having a lengthwise slot to accommodate the needle and two radiopaque rings in the guide in alignment with each other at opposite ends of the guide to orient the needle with the predetermined location and gaps in the rings matching the sides of the slot to enable the guide to be removed when the point of the needle is close to the predetermined location, and the like.

2. Brief Description of the Prior Art

Frequently, it is necessary to deposit dye, or medication to a specific location within a human (or animal) body. Often, it also is important to implant a substance within a body to identify the location of a tumor or the like that is to be removed through later surgery.

The surgical needles used in these procedures are supplied in a range of standard diameters. Further, the needles are relatively long, being sometimes five inches in length. The delivery ends of these needles, moreover, terminate in sharp points. These points are formed by bevelling the delivery ends at acute angles relative to their longitudinal axes. Other typical needles of this nature terminate in slender probes that protrude in a longitudinal direction from the bodies of the respective needles.

The problem that these long, slender needles present to the surgeon administering the medication, or taking other surgical action, is one of guidance to and alignment with the delivery location. For example, if medication is to be delivered to a specific place within a spine, advancing one of these beveled needles through (or past) one or more vertebra presents a major challenge to the surgeon. The nature of the point at the needle delivery end, as the needle is advanced through the patient, compels the needle to curve away from the delivery location. To cope with this tendency, surgeons have developed a technique of manually twirling the needle as it is advanced through the body and toward the delivery goal.

To assure that the delivery end of the needle continues on course to its predetermined location, the needle frequently is mounted in a radiopaque support that is external to the patient's body. To confirm the position of the needle relative to its destination, while reducing radiation exposure to the patient and to the surgeon, x-ray images are intermittently taken in two mutually perpendicular planes. Consequently, the needle can be advanced only in intermittent steps, rendering the entire procedure time consuming and fatiguing for the operating room staff This is a situation that is particularly aggravated when penetrating several inches of patient tissue.

Clearly, there is a need to improve needle guidance and provide surgeons with better control over the progress of the needle toward its predetermined destination.

### BRIEF SUMMARY OF THE INVENTION

These and other problems are, to a large extent, overcome through the practice of the invention. Illustratively, by using a guide with a needle accommodating slot in which the guide is radio-transparent (or radiolucent) to x-radiation, two radiopaque rings mounted within the guide and each being in longitudinal alignment at opposite ends of the guide enable the surgeon to visually align the needle with its destination in the patient.

By maintaining that alignment the surgeon can advance the needle continuously, in one steady motion, relying on the intermittent x-ray images only to confirm progress toward the needle's destination. In this manner radiation exposure is not increased while nevertheless significantly reducing the time taken by the procedure (and thereby reducing operating room staff fatigue) and greatly improving needle guidance accuracy. Further in this respect each of the rings has a gap, the gaps corresponding to the sides of the slot in the guide.

The two rings, in essence, form a sighting device in which the needle is visually aligned by the surgeon with its destination within the patient, the destination thereby forming a “target.” When the needle's delivery end is close to its destination, the slot in the guide permits the guide to be removed from the needle's shank in order to allow the surgeon to manipulate the needle through more extreme ranges of needle direction.

There are, moreover, several possible structures for mounting the guide relative to the patient. For example, a “C” shaped mount for supporting the needle and guide with a hand grip protruding from an end of the “C” a sufficient distance to protect the surgeon from excessive radiation exposure is preferred. Apparatus for mounting the guide directly on the x-ray apparatus or on other surfaces also can be used.

These and other features of the invention are described in the following detailed description of illustrative embodiments of the invention when taken with the figures of the Drawing. The scope of the invention, however, is limited only through the claims appended hereto.

### BRIEF DESCRIPTION OF THE SEVERAL VIEWS OF THE DRAWING

FIG. 1 is a plan view of a typical embodiment of the invention;

FIG. 2 is a transverse section of the embodiment of the invention shown in FIG. 1 and viewed in the direction of the arrows 2-2 of FIG. 1;

FIG. 3 is a transverse view of a modified embodiment of the invention shown in FIG. 1 with a needle positioned in it and as viewed from guide end 12;

FIG. 4 is a plan view in partial section of the embodiment of the invention shown in FIG. 1, positioned in a “C” mount;

FIG. 5 is a traverse view of the embodiment of the invention shown in FIG. 4;

FIG. 6 is a transverse section of the guide shown in FIG. 4 viewed in the direction of the arrows 6-6 of FIG. 4 that illustrates a further embodiment of the invention;

FIG. 7 is an exploded perspective view of still another embodiment of the invention;

FIG. 8 is a view of the embodiment of the invention shown in FIG. 7, when assembled, and seen in the direction of the arrows 8-8; and

FIG. 9 is a view of the embodiment of the invention shown in FIG. 7, when assembled, and seen in the direction of the arrows 9-9.

### DETAILED DESCRIPTION OF THE PREFERRED EMBODIMENT OF THE INVENTION

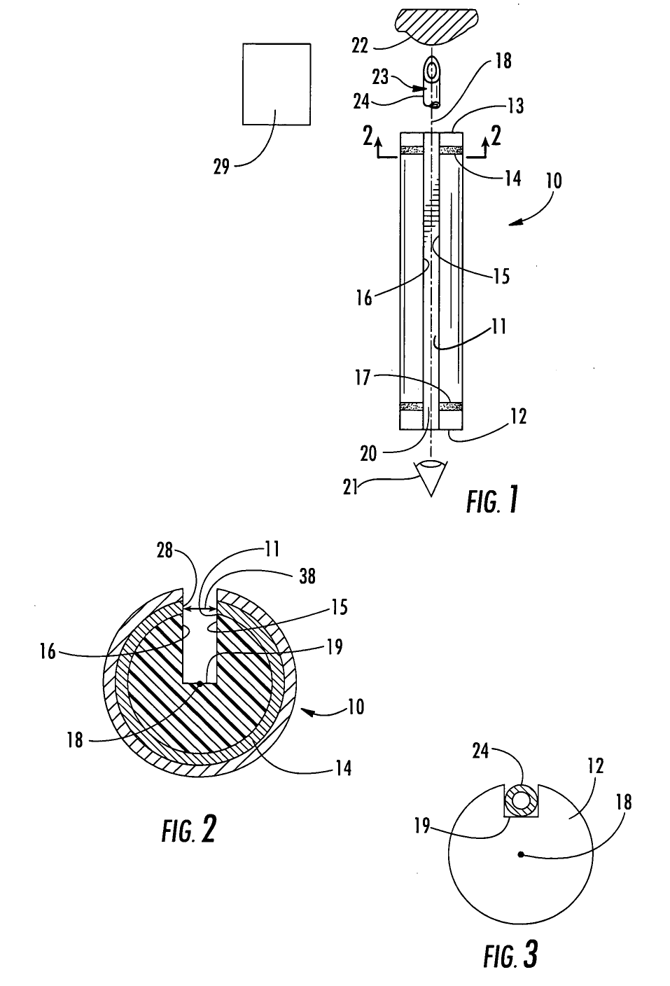

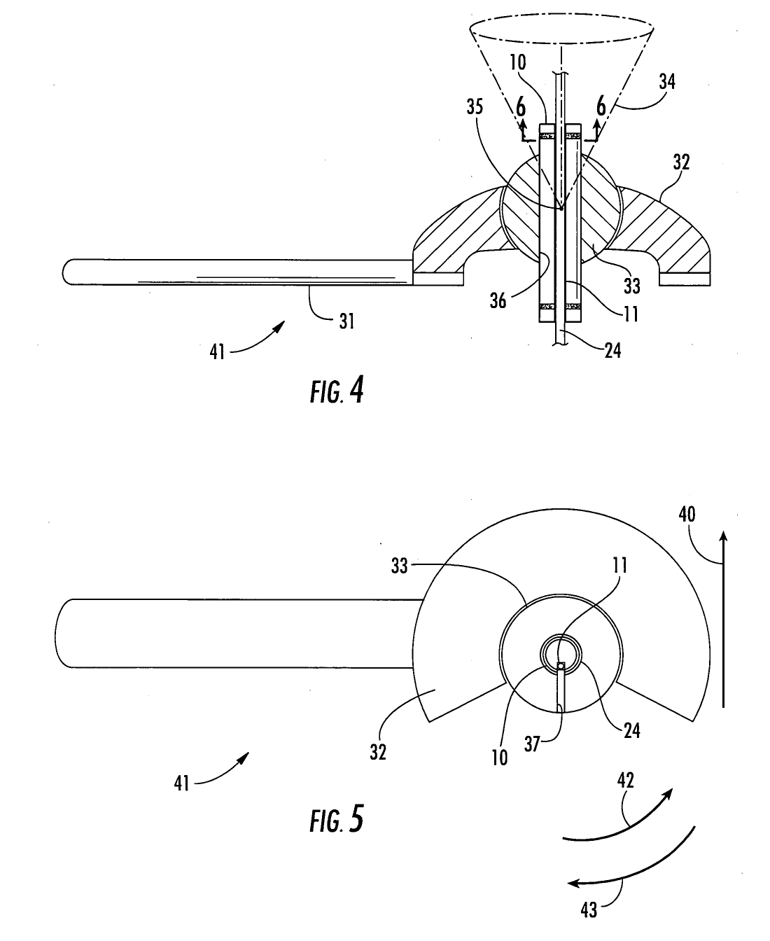

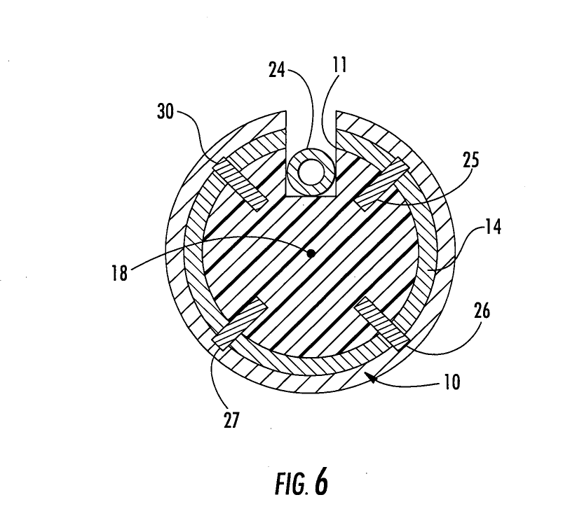

In accordance with the invention, FIG. 1 shows a plan view of a generally cylindrical guide 10; formed of a suitable radio-transparent material, that is, a material that does not create a pronounced x-ray image on an x-ray film or on an imaging screen or the like. FIG. 2 shows a lengthwise slot 11 formed longitudinally in the surface of the guide 10 of sufficient width and depth to accommodate a probe, or needle 24 (FIG. 3), selected, for example, from a full-range of hypodermic needles, e.g. at least 10 to 25 gauge hypodermic needles. The slot 11 (FIG. 2) is parallel with and as deep within the slot 11 as longitudinal guide axis 18, so that slot base 19 coincides with the longitudinal axis 18. Some embodiments of the invention, moreover, can have the slot base 19 spaced from either side of the centerline 18 as illustrated in FIGS. 3 and 6, with the base 19 of the slot 11 being spaced between the axis 18 and the upper side of the guide 10. As shown in FIG. 1, the guide 10 has a transverse end 12 at one lengthwise, or longitudinal extremity of the guide 10, and a transverse delivery end 13 at the other lengthwise extremity of the guide 10. Molded within the guide 10 toward the delivery end 13 is a circular ring 14 that is placed within the guide 10. The ring 14 is made from a suitable material, e.g. lead wire, that is radiopaque (a substance that produces a clear image on an x-ray viewing screen). A gap 28 (FIG. 2) is provided in the ring 14 that corresponds to slot sides 15 and 16 thereby leaving the slot 11 open to the surface of the guide 10. The center of the ring 14, moreover, is coincident with the axis 18 of the guide 10.

Spaced longitudinally from the delivery end 13 and close to the end 12 of the guide 10 another ring 17 (FIG. 1) of radiopaque material, and of similar construction to the ring 14, also is mounted within the guide 10. A gap 20 is formed in the ring 17 to provide an unobstructed and open channel for the slot 11. The ring 17 not only has a center (not shown in the drawing) that coincides with the slot axis 18, but also is of the same diameter and thickness as the ring 14 at the delivery end 13 of the guide 10.

In accordance with the invention, the two rings 14 and 17 are in careful register with each other in order to allow a surgeon's eye 21 to visually align the ring 14 at the delivery end 13 of the guide 10 with the ring 17 at the end 12 and with a delivery target 22 within the patient's body. By keeping the rings in alignment with the predetermined delivery target 22, the surgeon now can advance delivery end 23 of the needle 24 in a continuous and steady motion, relying only on an x-ray apparatus 29 for providing intermittent two-plane x-ray images to confirm the accuracy of the progress of the needle 24 toward the delivery target 22.

Attention now is invited to FIG. 6 which shows a further embodiment of the invention. Illustratively, the guide 10 in the delivery end 13 (FIG. 1) has the radiopaque ring 14 center (FIG. 6) coinciding with the longitudinal axis 18 of the guide 10. Radially disposed with respect to the axis 18 is an array of radiopaque angular indicia 25, 26, 27 and 30, mounted in the guide 10 so that the individual indicia 25, 26, 27 and 30 each are separated from its two adjacent indicia by 90°. The indicia 25 and 30, moreover, each being radially spaced 45° from the center 18 of the guide 10, and on opposite sides of the slot 11.

A similar arrangement (not shown in the Drawing) is provided for the end 12 (FIG. 1) of the guide 10. The indicia 25, 26, 27 and 30, when visually aligned with their corresponding counterparts mounted near the ring 17 in the delivery end 12 provide the attending surgeon with a clear indication of any angular deviation of the needle 24 relative to its predetermined orientation with respect to the delivery target 22.

In operation, and turning now to FIG. 4, a handle 31 is joined to a “C” shaped holder 32 both of which are radio-transparent. Centrally disposed within the arc of the holder 32 is a spherical bearing 33, also formed of radio-transparent material. The bearing 33 is seated in the arc of the holder 32 in a manner that enables the bearing 33 to be swept through a cone 34 with a conical apex angle of about 60° relative to center 35 of the bearing 33. A diametrical bore 36 is formed in the bearing 33 within which the guide 10 is snugly mounted. Further in this respect, a bearing slot 37 (FIG. 5) is provided in the bearing 33 that matches width 38 (FIG. 2) of the slot 11 in the guide 10.

Toward the end of the procedure, when the surgeon decides to remove the holder 32 and advance the needle 24 manually through the final distance to the delivery target 22 (FIG. 1), it is only necessary to turn the guide 10 to align the slot 11 with the bearing slot 37 (FIG. 5) and manually lift the entire assembly 41, that is the holder 32; the bearing 33; and the guide 10 in the direction of arrow 40. Upon drawing the assembly away in the foregoing manner, only the needle 24 remains in place between the hand of the attending surgeon (not shown) and the delivery target 22 (FIG. 1).

Naturally, the holder 32 can be dismounted to release not only the bearing 33, but also the entire holder assembly 41 for cleaning, sterilization and the like.

At the outset of the procedure, the guide 10 (FIG. 4) is inserted into the bore 36 in the bearing 33 and the needle 24 then is passed into the slot 11 in the guide 10.

The surgeon then manipulates the handle 31 with the attached holder assembly 41 over the patient and close to the delivery target 22 (FIG. 1). The surgeon turns the guide 10 in the direction of arrow 42 (FIG. 5) in order to temporarily close the slot 11 (FIG. 4) and thus selectively to lock the needle in place between the slot base 19 (FIG. 2) and the corresponding surface of the bore 36 (FIG. 4).

The surgeon then presses the needle 24 (FIG. 1) into the patient's body and visually aligns the rings 14 and 17 with the delivery target 22 within the patient's body. The needle, so aligned, then is advanced steadily toward the delivery target 22, taking advantage of the intermittent two-plane x-ray images to confirm (or correct) the position of the needle 24 relative to the delivery target 22. As the needle 24 approaches the delivery target 22 and the surgeon decides that more freedom of movement for the needle 24 is required, the guide 10 (FIG. 5) is turned manually in the direction of arrow 43 to shift the slot 11 and the needle 24 nested within the slot 11 away from the confines of the bore 36 within the bearing 33. In this orientation the holder assembly 41 can be lifted away from the needle 24 by raising the holder assembly 41 in the direction of the arrow 40 as described above.

Should the guide 10 be equipped with the indicia 25, 26, 27 and 30 (FIG. 6) the attending surgeon also will be assisted in maintaining the angular orientation of the needle 24 relative to the delivery target.

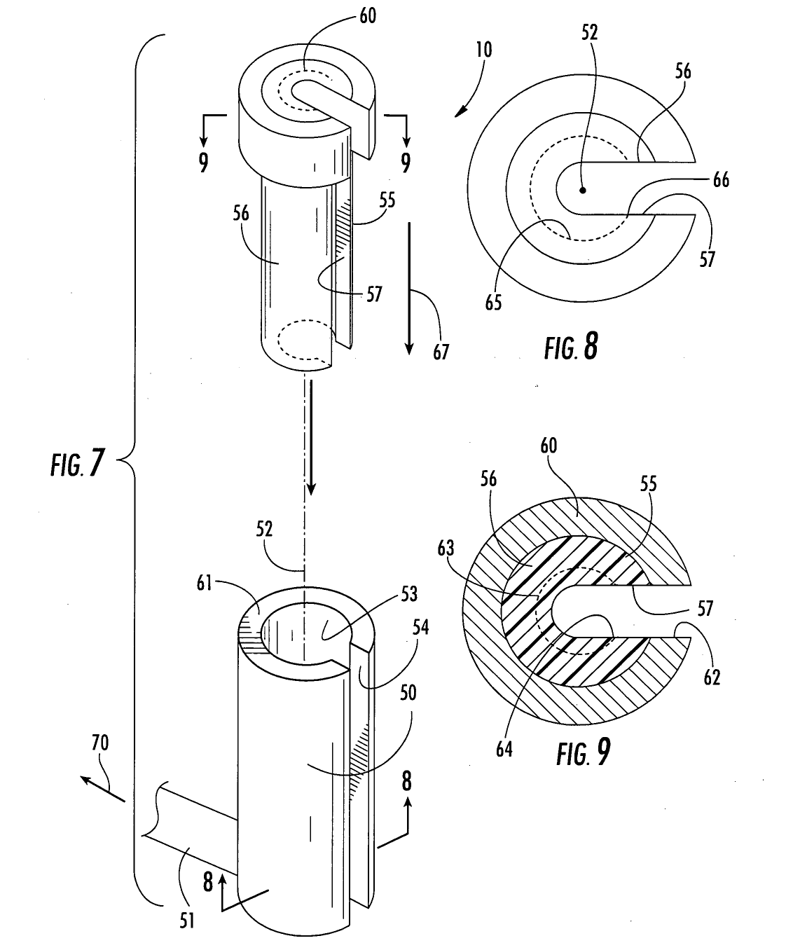

Attention now is invited to FIG. 7 which shows the guide 10 as a two piece assembly. Thus, the guide 10 has as a first piece a generally cylindrical and radio-transparent support 50 mounted on a stabilizing device, or handle 51 that protrudes laterally from the support 50 and is generally perpendicular to longitudinal centerline 52 for the support 50.

As shown, a longitudinal bore 53 is formed in the support 50. Further in accordance with the invention, a lengthwise slot 54 also is provided in the support 50 in communication with the bore 53. The slot is in alignment with the longitudinal centerline 52 for the support 50. The support slot 54 is at least as wide as the diameter of the needle 24 (FIG. 3) to enable the guide 10 (FIG. 7) and the handle 51 to be removed when the slot 54 and slot 57 are in transverse alignment as illustrated with respect to the embodiment of the invention shown in FIGS. 4 and 5.

The second piece in the guide 10 is the radio-transparent insert 55 in which generally cylindrical stem 56 is sized to fit snugly with the support bore 53.

Turning now to FIG. 9, it can be seen that the insert 55 is attached to a collar 60 that has an outside diameter sufficient to rest upon an adjacent transverse end 61 (FIG. 7) of the support 50. As shown, the collar 60 (FIG. 9) is secured to the uppermost end of the stem 56 in order to be rotatable with the collar 60 as a single unit. Note further that the slot 57 in the stem 56 registers with a corresponding slot 62 in the collar 60. The collar slot 62, moreover, preferably matches the width of the lengthwise slot 54 (FIG. 7) in the support 50. The collar 60 also has, within its structure a radiopaque ring 63. A gap 64 is provided in the ring 63, the ends of the gap 64 being coterminous with the sides of the slot 62 in the collar 60, and the center of the ring coinciding with the longitudinal centerline 52 for the guide 10.

Attention now is invited to FIG. 8 which illustrates a radiopaque ring 65 in the lower end portion of the stem 56. Although the center of the ring 65 also coincides with the centerline 52 of the guide 10, thus aligning the rings 63 and 65 longitudinally with respect to the centerline 52, the diameter of the stem ring 65 is appreciably smaller than that of the collar ring 63. Further with respect to the stem ring 65, it will be seen in the drawing that a gap 66 is formed in the ring 65. The sides of the gap 66 in the ring are generally equal to the width of the sides of the stem slot 57 and the slot 54 in the support 50.

In operation, the collar 60 (FIG. 7) and the attached stem 56 are inserted in the direction of arrow 67. The collar is turned in either direction to align the stem slot 57 with the support slot 54. An appropriate needle (not shown in FIG. 7) is then placed in the slot 57 within the now assembled guide 10. Next, the collar is turned again to eliminate the alignment between the slots 54 and 57 and selectively lock the needle in the stem slot 57.

The x-ray apparatus is intermittently activated to permit the attending surgeon to orient the rings 63 and 65 with respect to the delivery target 22 (FIG. 1). The needle then is pressed into the patient and advanced steadily and continuously toward the point of delivery by keeping the rings 63 and 65 always in alignment. The brief activations of the two-dimensional x-ray equipment, moreover, permit the attending surgeon to confirm the progress of the needle toward the point of delivery. As the point of delivery is closely approached, and the surgeon requires greater freedom of action with the needle's orientation, the collar 60 (FIG. 7) is once more turned to align the slots 54 and 57 in the guide 10 with each other. Once aligned the entire assembly of the guide 10 can be removed by shifting the handle 51 in the direction of arrow 70.

Further methods and apparatus for stabilizing and supporting the guide 10 relative to the predetermined location can include the x-ray machine structure, or the patient or other suitable support. Illustratively, a holder assembly can be provided that will rest directly on the x-ray apparatus. Accordingly, the practice of the invention enables a surgeon to swiftly and accurately advance a hypodermic needle to a predetermined position within a body without incurring any additional radiation exposure to the patient and operating room personnel. And, perhaps, because the invention permits a continuous advance of the needle 24 (FIG. 1) to the delivery target 22 this entire procedure now can be accomplished more swiftly, thereby reducing the overall radiation exposure to the patient, the surgeon and the operating room personnel.

What is claimed is:

1. A mechanism for aligning a probe with a predetermined location within a body, comprising a radio-transparent guide having a lengthwise slot formed in the surface thereof, said slot having a width that is at least equal to the width of the probe, a pair of radiopaque rings spaced from each other toward opposite lengthwise ends of said guide, each of said rings having gaps formed in the respective circumferences thereof, said gaps each being essentially as wide as said slot and corresponding to said slot sides for aligning said rings with the predetermined location in order to continuously and accurately advance the probe toward the predetermined location.

2. A mechanism according to claim 1 wherein said guide further comprises a generally cylindrical shape having a lengthwise slot formed in the surface of said guide, said slot having a width sufficient to accommodate a range of the probes and being aligned with said cylindrical guide axis.

3. A mechanism according to claim 1 further comprising radiopaque indicia angularly disposed within said guide and generally perpendicular to the circumference of at least one of said radiopaque rings.

4. A mechanism according to claim 1 further comprising two sets of said radiopaque indicia each of said sets of indicia being generally perpendicular to the circumference of a respective one of said radiopaque rings.

5. A mechanism according to claim 1 further comprising a radio-transparent holder for the mechanism, said holder having a radio-transparent bearing selectively seated in said holder, said bearing having a bore formed therein for snugly receiving said radio-transparent guide, a slot formed in said bearing that corresponds to said guide slot for selective alignment with said guide slot to enable said holder to disengage the probe.

6. A mechanism according to claim 5 wherein said holder has a “C’ shape, said bearing having a generally spherical shape for being selectively mounted in said holder.

7. A mechanism according to claim 6 further comprising a handle attached to one side of said “C” shape for manually supporting the mechanism relative to the predetermined location.

8. A mechanism according to claim 1 further comprising means for stabilizing said probe relative to the predetermined location.

9. A mechanism according to claim 1 further comprising an x-ray apparatus for the intermittent display, in at least two dimensions of the predetermined location, said radiopaque rings enabling the probe to remain in alignment with the predetermined location when said x-ray apparatus fails to display the position of the probe relative to the predetermined location.

10. A method for continuously advancing a probe toward a predetermined location within a body comprising the steps of activating and deactivating a two dimensional x-ray display of the position of the probe relative to the predetermined location, placing the probe in a slot in a radio-transparent guide, pressing the probe into the body, visually aligning a pair of radiopaque rings in said radio-transparent guide with the predetermined location, continuously advancing the probe toward the predetermined location during activation and deactivation of the x-ray apparatus, turning the slot in the guide in order to release the probe from the guide as the probe is close to the predetermined location.

11. A mechanism according to claim 1 wherein said rings further comprise said rings being each of different diameter.

12. A mechanism according to claim 1 wherein said rings further comprise said rings being each of the same diameter.

13. A mechanism for aligning a probe with a predetermined location within a body comprising a radio-transparent guide with a support having a longitudinal support bore formed in said support and a lengthwise support slot formed in said support in communication with said support bore, said support slot being at least as wide as the probe, an insert having at least a lengthwise portion thereof sized to fit snugly within said support bore and having an insert slot that corresponds to and is in alignment with said support slot to accommodate the probe, and a pair of radiopaque rings spaced from each other at opposite longitudinal ends of said insert and in alignment with said longitudinal support bore.

14. A mechanism according to claim 13 wherein said radiopaque rings further comprise different diameters.

15. A mechanism according to claim 13 further comprising said rings being of the same diameter.

16. A mechanism according to claim 13 further comprising a collar secured to one end of said insert and having a collar slot formed therein, said collar slot matching said support slot and said insert slot and being in alignment with said support slot and said insert slot.

17. A mechanism according to claim 16 wherein said support, said insert and said collar further comprise said radio-transparent guide for selective rotation together.
